(19) United States
(12) Patent Application Publication
(10) Pub. No.: US 2026/0279536 A1
(43) Pub. Date: Sep. 17, 2026

(54) PRECISION DISEASE PREVENTION AND LONGEVITY ENGINE TAILORED TO INDIVIDUAL GENETIC AND ENVIRONMENTAL PROFILES

(71) Applicant: Vydiant, Inc., Folsom, CA (US)

(72) Inventors: Jim Kaput, Madison, WI (US); Ray Scheid, Rochester, MN (US); Edith Coronado, Bussigny (CH); Parismita Bharali, Germantown, MD (US); Albin Kurian, Ernakulam District (IN); Dennis Minn, Jericho, NY (US); Terry Carlone, Sacramento, CA (US)

(21) Appl. No.: 19/563,962

(22) Filed: Mar. 11, 2026

Related U.S. Application Data
(60) Provisional application No. 63/770,882, filed on Mar. 12, 2025.

(51) Int. Cl. G16H 20/10; G16B 5/30; G16B 30/00; G16H 10/60; G16H 20/30; G16H 20/60; G16H 50/20; G16H 70/60

(52) U.S. Cl. CPC G16H 20/10; G16B 5/30; G16B 30/00; G16H 10/60; G16H 20/30; G16H 20/60; G16H 50/20; G16H 70/60

(57) ABSTRACT

An integrated personal health system, method and device for development of a precision disease prevention and longevity engine for the continuous, individualized optimization of human healthspan and extension of lifespan through real-time multiomic data acquisition, dynamic biological modeling, and adaptive lifestyle and other intervention generation tailored to individual genetic profiles is described herein.

### CROSS-REFERENCE TO RELATED APPLICATION(S)

This application claims the benefit of U.S. Provisional Patent Application Ser. No. 63/770,882, filed Mar. 12, 2025, and titled “PRECISION DISEASE PREVENTION AND LONGEVITY ENGINE TAILORED TO INDIVIDUAL GENETIC PROFILES,” which is hereby incorporated by reference in its entirety for all purposes.

### FIELD OF THE INVENTION

The present invention relates to the field of personal health. Specifically, the present invention relates to the extension of human healthspan through systems, methods, and devices for development of adaptive lifestyle and other interventions.

### BACKGROUND OF THE INVENTION

Health and disease research over the past one hundred years was largely based on reductionist experimental approaches which tested a single variable while holding all other variables constant. This methodology served science and society well, dramatically improving health and longevity. In spite of these advances, multifactorial diseases such as obesity, diabetes, and other chronic diseases have become near-epidemics across the world, indicating that reductionistic strategies provide incomplete solutions. The critical concept overlooked from health research and applications is that health results from interactions of multiple in-the-body molecules and processes and many external factors that include nutrition/diet, physical activity/exercise, social activity, environment, stress, sleep, behavior and others, while disease results from dysfunctions of one or more interacting processes among these scales. Thus, many genetic studies overlook lifestyle variables that can alter disease progression or affect Polygenic Risk Score (PRS) calculations. The interaction of the internal and external processes produces emergent (unexpected) properties of the separate macro and micro systems. However, applying these concepts experimentally or directly to the real world has not happened: economic and other disincentives and challenges have deterred integration of knowledge across these different sectors. In addition, it is only recently that advances in artificial intelligence and machine learning, and increases in “big data” computing capacity, and advances in polygenic and specific pathway risk analysis have made it possible to harness the large volumes of data involved in aggregating and analyzing the immensely complex matrices of information interconnected across multiple scales and multiple morbidities in human biological systems.

Existing medical systems are organized around disease detection and treatment rather than the upstream biological processes that produce disease over decades. Current preventive medicine approaches rely on population-average risk statistics that fail to capture the profound individual variation in disease susceptibility, biological aging rate, and intervention response driven by genetic architecture and other factors. Consumer genomic tools provide static genetic risk information without mechanistic intervention pathways. Wearable health devices generate biometric data without integration into causal biological models. Biological age assessment tools operate episodically without connection to adaptive intervention systems.

### SUMMARY OF THE INVENTION

A genetic profile-based disease intervention system, method and device including a computational pipeline that systematically identifies transcription factors (TFs) regulating disease or other condition-associated genes (e.g., diabetes-associated genes), maps their interactions with small molecules (e.g., ligands), and links these small molecules to nutritional/diet components, physical activity/exercise, social activity, environment, culture, stress, sleep, behavior and other lifestyle variables. Thus, the genetic profile-based disease intervention system, method and device provides the benefit of enabling the development of precision lifestyle interventions tailored to individual genetic profiles.

The systems, methods and devices provide a precision disease prevention and longevity engine comprising three integrated components: (1) a system for multiomic and physiological data acquisition and integration; (2) a method for individualized biological modeling, disease risk trajectory prediction, and intervention optimization; and (3) a device architecture enabling real-time closed-loop biological monitoring and adaptive intervention delivery.

The system is distinguished from prior art by several novel and non-obvious features. First, it constructs a continuously calibrated digital persona model of each individual's specific physiology, enabling counterfactual intervention simulation and personalized response prediction that transcends population-average evidence.

Second, the system employs healthspan and biological aging as a continuous, multi-system outcome metric rather than disease-centric diagnostic thresholds, enabling intervention and measurement at preclinical timescales.

Third, the system integrates a causal biological knowledge graph with a reinforcement learning and reasoning optimization engine to identify synergistic intervention combinations across the full multi-domain lifestyle space, explicitly modeling pathway interactions and genetic response heterogeneity.

Fourth, the system operates a multi-modal, continuous anomaly detection system capable of identifying subclinical disease signals across molecular, physiological, environmental, and behavioral data streams years to decades before conventional clinical detection.

Fifth, the system employs a federated learning architecture that preserves individual data sovereignty while enabling population-scale scientific discovery from aggregated anonymized response patterns, generating novel gene-environment interaction discoveries as a continuous byproduct of system operation.

The systems, methods and devices convert individual genetic architecture from a source of statistical risk estimates into a precision instruction set for biological optimization, uniquely operationalizing the frontier of molecular aging science at the level of individualized daily intervention in a continuously improving, causally reasoning closed-loop system.

### BRIEF DESCRIPTION OF THE DRAWINGS

FIG. 1 illustrates a diagram of a data-driven resource that has been developed using public domain data to link Transcription Factors (TFs) to genetic risk, environmental exposures, and disease outcomes according to some embodiments.

FIG. 2 illustrates, as an example of one disease, a flowchart of a method of precision disease prevention for diabetes according to some embodiments.

FIG. 3 illustrates a block diagram of an exemplary computing device configured to implement the precision disease prevention method according to some embodiments.

FIG. 4 illustrates a diagram of a precision disease prevention system according to some embodiments.

FIG. 5 illustrates a diagram of a precision disease prevention device architecture according to some embodiments.

FIG. 6 illustrates a diagram of a precision disease prevention framework according to some embodiments.

### DETAILED DESCRIPTION OF THE PREFERRED EMBODIMENT

No existing system integrates these capabilities into a unified, continuously learning, causally reasoning and teaching engine that operates upstream of disease at the level of individual molecular biology. A critical unmet need exists for a system that treats each individual as a biological sample size of one, models their complete physiology, medical history, family history, culture, environment, physical activity, social engagements, environment, stress, sleep and mental health as an integrated dynamic system, and generates precision interventions calibrated to their specific genetic architecture and observed biological responses.

An integrated personal health system, method and device for development of a precision disease prevention and longevity engine for the continuous, individualized optimization of human healthspan and extension of lifespan through real-time multiomic data acquisition, dynamic biological modeling, and adaptive lifestyle and other intervention generation tailored to individual genetic profiles is described herein.

Trained AI-driven data mining is utilized to map environmental ligands/activators to transcription factors regulating genes involved in a selected disease/condition (e.g., Type 2 Diabetes Mellitus (T2DM)) pathways, identified through published partitioned-polygenic risk analysis, identify nutrients, physical activities, and stressors that impact these molecular pathways, accounting for individual variations. Although the description uses the example of diabetes as the selected disease/condition, it is understood that other types of diseases/conditions and the related genes are able to be selected.

Many genetic studies overlook lifestyle variables that can alter disease progression or affect Polygenic Risk Score (PRS) calculations, although there are exceptions. For instance, different studies have used 5 and 159 Single Nucleotide Polymorphisms (SNPs) to construct a PRS that accounted for diet quality, fat, carbohydrate, and total energy intake on β-cell dysfunction, impaired insulin synthesis, obesity, body fat distribution or fat mass, lipid/hepatic metabolism, and global diabetes pathways. Another PRS study analyzed the combination of five modifiable lifestyle factors (body mass index (BMI)<25, high diet quality, regular exercise>150 minutes/week of moderate or 75 minutes/week of vigorous, moderate alcohol consumption, and never smoking) and 59 SNPs involved in diabetes. More recently, scores of GWA studies conducted in different multi-ethnic populations have identified 650 variants associated with total polygenic risk applicable to diverse genetic backgrounds. Higher genetic risk scores are associated with an increased risk of T2DM, while lower scores are linked to a reduced risk. Notably, adherence to optimal modifiable factors was found to reduce the risk of T2DM, even among individuals with higher genetic susceptibility.

TABLE 1
Transcription Factors and Nutrients

| | Food | Transcription | |
|---|---|---|---|
| Nutrient | Example | Factor | Pathway |
| Carbohydrate | Multiple | ChREBP/MLXIPL | Lipid metabolism |
| | | CREB3 family | |
| | | FOXO | |
| Resveratrol | Grapes, | PPARγ | Fat Metabolism |
| | Red Wine | | |
| Curcumin | Turmeric | NF-κB | Anti-inflammatory |
| Sulforaphane | Broccoli | NRF2 | Antioxidant Defense |
| Genistein | Soy | ERβ, ATF3 | Hormone Signaling |

TABLE 2
Transcription Factors, Physical Activity, and Stress

| Transcription Factor | Activator/Ligand | Role |
|---|---|---|
| PPARγ (Peroxisome Proliferator- | Exercise-induced generation of | Enhances mitochondrial biogenesis, |
| Activated Receptor Gamma) | endogenous ligands | improves metabolic health |
| NRF2 (Nuclear Factor Erythroid | Oxidative stress | Regulates antioxidant genes, protects |
| 2-Related Factor 2) | | against oxidative damage |
| ATF3 (Activating Transcription | Physiological stress, exercise | Modulates immune function, involved in |
| Factor 3) | | exercise-induced transcriptional changes |
| HSF1 (Heat Shock Factor 1) | Elevated temperature, cellular | Induces heat shock proteins, protects |
| | stress | against stress-induced protein damage |
| NFAT (Nuclear Factor of | Calcium ions | Regulates muscle fiber transformation, |
| Activated T-Cells) | | adapts muscles to endurance exercise |
| NR4A1 (Nuclear Receptor 4A1) | Fatty acids, stress, growth factors, | Regulates gene expression in response to |
| | mechanical agitation | physiological stimuli and exercise |

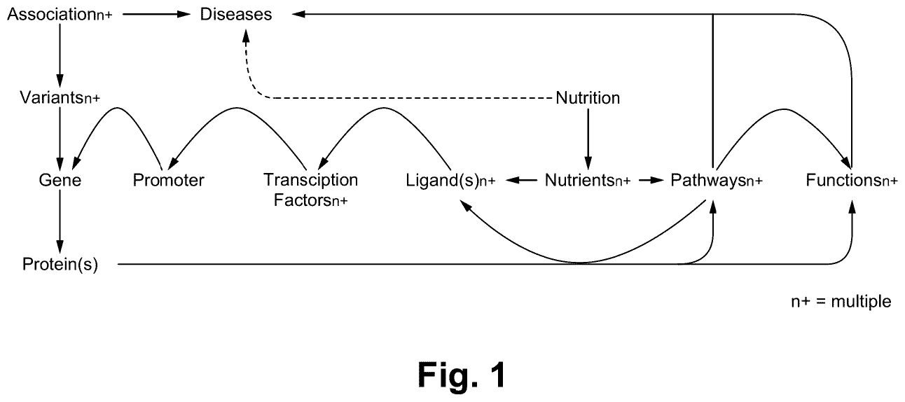

Since Genome-Wide Association Studies (GWAS) are expensive to conduct and lifestyle data difficult to collect from thousands to tens of thousands of participants, a data-driven resource has been developed using public domain data to link Transcription Factors (TFs) to genetic risk, environmental exposures, and disease outcomes, as shown in FIG. 1. Diet-derived ligands activate gene expression (Table 1) and activator/ligands have been identified for other environmental factors (Table 2). Although signaling pathways such as AMP-activated protein Kinase (AMPK) activation, mammalian Target Of Rapamycin (mTOR) and Nuclear Factor kappa B (NF-kB) inhibition, chromatin remodeling, and epigenetic mechanisms also contribute to regulating gene expression, TFs usually have the largest effect on gene regulation. Specifically, a computational pipeline systematically identifies TFs regulating diabetes-associated genes, maps their interactions with small molecules (ligands), and links these ligands to nutritional components, physical activity, and emotional stress. This enables the development of precision lifestyle interventions tailored to individual genetic profiles.

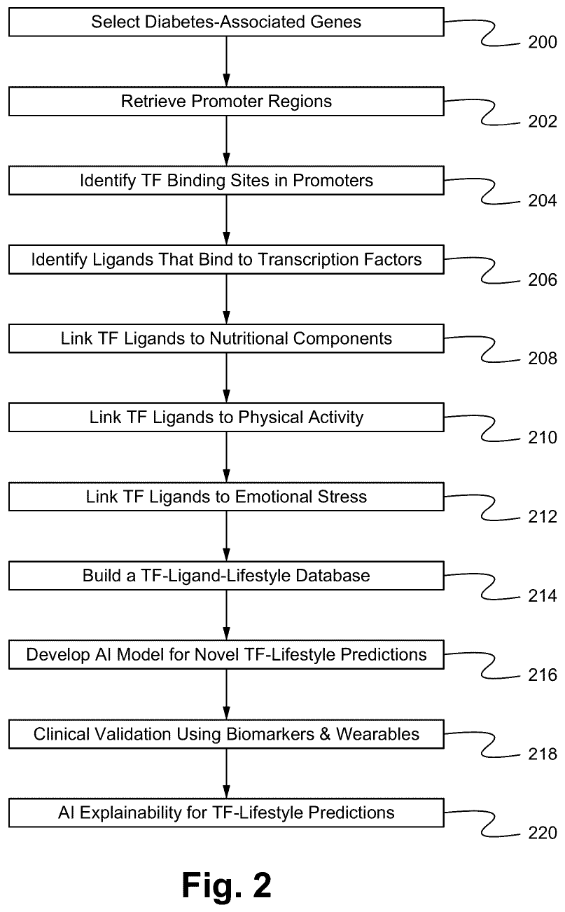

FIG. 2 illustrates, as an example of one disease, a flowchart of a method of precision disease prevention for diabetes according to some embodiments. In the step 200, diabetes-associated genes are selected. GWAS studies, PRS, or literature mining are used to identify diabetes-relevant genes. In the step 202, promoter regions are retrieved. Promoter regions (e.g., −2 kb to +500 bp from Transcription Start Site (TSS)) are extracted using genomic databases such as Ensembl BioMart and UCSC Genome Browser. In the step 204, TF Binding Sites are identified in promoters. Chromatin immunoprecipitation sequencing (ChIP-seq) and motif-based analysis are used to detect TF binding in promoter regions. In the step 206, ligands that bind to TFs are identified. Databases are queried for experimentally validated TF-ligand interactions. In the step 208, TF ligands are linked to nutritional components. Food sources containing TF ligands are identified using food compound databases such as FooDB, NPASS, USDA FoodData Central, and Langual. In the step 210, the TF ligands are linked to physical activity. Exercise-induced molecular changes that regulate TF activity are identified (e.g., in Reactome, GeneCards, Exercise Gene Network (ExGen). In the step 212, TF ligands are linked to emotional stress. Stress-related hormones and neurotransmitters affecting TF activity are identified (e.g., in PsychENCODE, Gene Ontology (GO), AI-Based Literature Mining (GPT-5.2o or the latest versions)). In the step 214, a TF-ligand-lifestyle database is built. A structured database is developed for TF-ligand-lifestyle relationships. PostgreSQL, ChEMBL, DrugBank, FooDB, PubChem are exemplary databases. In the step 216, an AI Model for novel TF-lifestyle predictions is developed. A machine learning model integrating gene expression, ligand structure, and lifestyle impact is trained. TensorFlow, XGBoost, RNA-seq (GTEx), Molecular Docking Simulations are exemplary databases. In the step 218, clinical validation is performed using biomarkers and wearables. AI predictions are validated using real-world biomarker data such as Continuous Glucose Monitor (CGM), Heart Rate Variability (HRV), and blood biomarkers. UK Biobank, NIH All of Us, GTEx, Framingham Heart Study are exemplary databases. In the step 220, AI explainability is implemented for TF-lifestyle predictions. SHAP and LIME are used to interpret model predictions and improve AI transparency. In some embodiments, fewer or additional steps are implemented. In some embodiments, the order of the steps is modified.

In some embodiments, the pipeline starts with identifying a comprehensive list of disease/condition-relevant genes (e.g., diabetes-relevant genes but applicable to any chronic disease) using the large database generated from hundreds of GWAS results, analysis of consistent variants linked to the condition by those partitioning into pathways, and using literature mined with a pipeline (e.g., OneHealth pipeline) to provide guidance personalized to the individual. The strategy leverages existing public domain databases to identify TFs that regulate genes implicated in chronic disease (e.g., T2DM). Promotor regions (e.g., +2 kb to +500 bp from the transcription start site) are able to be found using Ensembl BioMart and UCSC Genome Browser and are able to be confirmed using ChIP-seq and motif-based analyses based from the ENCODE database (for TF occupancy data), JASPAR (for TF binding motifs), and ChEA3 (for experimentally validated TF interactions). The MEME Suite's FIMO tool is able to be used to scan extracted sequences for TF motifs. To systematically identify TF-ligand interactions, several bioactivity and drug-target interaction databases such as ChEMBL, DrugBank, PubChem BioAssay, and BindingDB are able to be used to extract information on small molecules with experimentally validated binding affinities to TFs. The Protein Data Bank (PDB) is able to be used to confirm TF-ligand interactions.

TF-ligand interactions are subsequently mapped specific to nutrients, physical activity, emotional stress, and/or social and environmental variables which alter the expression of genes involved in the chronic disease. A current dictionary (which used, for example, USDA FoodData Central, Langual International Food Description, and/or Vydiant-added components) includes 5,942 food-, nutrient-, polyphenol-, and supplement-terms. The number of terms in the OHLFHdb is currently 4,248 (that is, not every nutritional component in the dictionaries have been studied). Nevertheless, the dictionary is able to be supplemented with FooDB and the Natural Product Activity and Structural Similarity Database, food additives/preservatives, and validated and experimental biomarkers. Potential dietary ligands (e.g., omega-3 fatty acids that bind to peroxisome proliferator-activated receptor gamma (PPARG)) are mapped to specific foods (e.g., flaxseeds, salmon) using the various food databases, although none of these resources has complete data on every food.

Exercise is a key modulator of transcription factor activity, which influences metabolic health and insulin sensitivity. Several TFs responsive to physical activity have been identified (Table 2) from Pubmed searches. The list is expanded using GeneCard's Exercise Gene Network (ExGen) along with pathway databases. Chronic stress has been shown to act through stress-responsive TFs (Table 2). PsychENCODE, Gene Ontology (GO) and Reactome are able to be used to identify potential TFs responsive to stress. Insights from these steps are able to be used to recommend personalized stress-management strategies (e.g., mindfulness, sleep hygiene) to mitigate TF-driven metabolic dysregulation.

The results from the pipeline steps described above are added to a knowledge base (e.g., OneHealth Knowledge base) with structured information on TFs, ligands, and lifestyle factors. A deep learning model is trained to predict novel TF-lifestyle relationships using multi-model data inputs from ChIP-seq, ligand features (such as binding affinities), and lifestyle data of nutrient composition, exercise types, and stress. The results of this pipeline add molecular details to the directional relationships between these lifestyle factors and disease, health benefits, and mental health terms already in the OHLFHdb. TensorFlow and XGBoost are used for the classification of the data and explore SHAP and LIME explainability models for making interpretable AI predictions. The model is fine-tuned using real-world biomarker data and the TF-lifestyle interactions that have been published.

The accuracy and clinical relevance of the TF-lifestyle predictions is tested using real-world physiological data obtained from the intervention study and from data in the UK Biobank, NIH All of Us, and Genotype-Tissue Expression (GTEx) databases. The study is able to acquire Continuous Glucose Monitoring (CGM) that assesses the impact of lifestyle interventions on glucose metabolism and metabolic panel blood-based biomarker data. Some participants will have wearables that captures heart rate variability.

Although the OHLFHdb is currently in version 4, the OHLFHdb is expanded to include the data consolidated by the pipeline. FastAPI-based REpresentational State Transfer (REST) API is developed for access to the TF-ligand-lifestyle data. This API is able to feature endpoints for retrieving TFs associated with a given gene, identifying ligands that regulate specific TFs, and generating personalized lifestyle recommendations based on AI predictions. A current lifestyle factor-health database matches evidence-based guidance personalized to an individual's age, sex, lifestyle, and condition, but this is able to be enhanced by providing guidance based on total and partitioned polygenic risk scores. The API is able to be employed on AWS Lambda (or current cloud infrastructure), enabling seamless integration into digital health platforms and the OneHealth Go app.

The pipeline system, method and device described herein represent a novel approach to precision medicine, linking genetic risk factors to actionable, evidence-based interventions. The pipeline system, method and device are able to enable personalized dietary, exercise, and stress management guidance based on genetic profiles, integrate CGM and biomarker data for real-world validation of TF-driven interventions, and provide an AI-powered guidance support system for optimizing metabolic health. The pipeline system, method and device combination are able to incorporate systems biology, bioinformatics, and AI-driven insights to fill a critical gap in translating genetic risk into lifestyle-based disease prevention. The pipeline system, method and device generate a scalable, data-driven framework that can be widely adopted for precision nutrition, metabolic health improvements, and personalized wellness interventions. By restricting the generative algorithms to searching the pre-formed knowledge base, hallucinations are limited, if not eliminated, and the guidance is more comprehensive and reproducible.

The methodology utilizes a specialized machine learning system, based on Natural Language Processing (NLP) and recent and ongoing development of multi-hop reasoning methodologies, to accurately identify and extract relationships between lifestyle factors and health terms, including medical conditions, health benefits, and mental health indicators.

The system is trained using a meticulously curated corpus of human-annotated data including individual sentences derived from biomedical text. Domain experts explicitly label the relationship between a lifestyle factor (e.g., diet, physical activity, or behavioral factors) and a health term using predefined relationship types, such as positive effect, negative effect, no effect, or no relationship. This specialized dataset is used to fine-tune a Large Language Model (LLM), enabling the model to learn and generalize the linguistic, semantic, and contextual patterns that define factor-health outcome relationships.

Once trained, the model is deployed to process extensive biomedical literature, focusing on sentence level text sourced from over 44 million PubMed abstracts and PMC articles, which are updated and expanded on a regular basis. The fine-tuned model efficiently identifies, classifies, and extracts factor-health term relationships at scale. This process generates structured relationship data that is integrated into a knowledge base for downstream analysis and inference.

A machine learning based NLP and recent algorithms for generative processing systems trained on human-annotated data is used to extract structured relationships between diverse entities and concepts from unstructured text. The trained model is then applied at scale to large text corpora, such as scientific and technical literature, to automatically identify, classify, and extract relationships for inclusion in the structured knowledge base.

The system starts by gathering multiple streams of personal data: DNA from a saliva, cheek swab, or blood sample is sequenced or analyzed on variant-feature arrays to identify variants across hundreds of thousands of genetic loci. These include Single Nucleotide Polymorphisms (SNPs), chromosome positions, copy number variants or rate variations, and epigenetic markers that influence how genes are expressed in response to diet, exercise, stress, and sleep and how the variants are linked to diseases or traits.

Continuous physiological monitoring via wearables captures real-time biometrics: heart rate variability, sleep patterns, blood glucose (gathered from continuous glucose monitors), cortisol rhythms, activity patterns, body temperature, and SpO₂. These signals become the phenotypic fingerprint that is compared against the genetic baseline.

Behavioral and environmental inputs come from apps, food logging, location data, cultural data, air quality sensors, and self-reported mood and stress levels.

An analytical engine is the core intelligence of the system, operating in several layers: pharmacogenomic and nutrigenomic mapping identifies how specific genetic variants affect metabolism. For example, variants in the MTHFR gene affect folate metabolism and methylation; FTO and MC4R variants influence obesity risk and appetite regulation; ACTN3 variants affect fast-twitch versus slow-twitch muscle fiber composition, shaping optimal exercise types; CYP1A2 determines how quickly a person metabolizes caffeine or may activate precarcinogens. The genetic variants explain only a small percentage of the respective phenotypic variability in and between individuals.

Polygenic risk scoring increases reliability of predicting total lifetime risk by aggregating thousands of small-effect genetic variants into composite risk scores for conditions such as cardiovascular disease, type 2 diabetes, depression, or certain cancers. These are not deterministic—they inform probability landscapes that lifestyle can meaningfully shift. The variants identified by the analysis are then clustered and mapped to specific metabolic pathways contributing to disease incidence and/or outcome. This step identifies the relative contribution of metabolic, immune, hormonal or other processes to the condition providing insights into specific foods, nutrients, bioactives, and physical activities that could reduce the effect of the genetic contribution to the condition.

Machine learning models then integrate genetic data with the continuous biometric stream to identify personal response curves—how an individual's glucose responds to a carbohydrate load, how an individual's HRV recovers after a given sleep duration, how an individual's inflammatory markers (tracked via periodic blood panels) respond to different exercise intensities. Over time the system learns that what is true for the population average does not apply to a discrete individual.

Digital persona modeling is the more advanced version of this: a computational simulation of each individual's physiology that can run “what if” scenarios before that individual makes lifestyle changes, predicting downstream effects. The approach uses the ever-growing amount of population level data that provides ranges of possible responses used in modeling the trajectory of an individual.

Rather than generic recommendations, the system generates precision protocols: nutrition interventions might specify not just macronutrient ratios but meal timing, specific micronutrient targets, and foods to avoid based on an individual's genetic metabolic tendencies and real-time glucose response data. For example, an individual with APOE4 variants may need higher amounts of omega-3 and increased emphasis on Mediterranean diet recommendations given their elevated Alzheimer's and cardiovascular risk. Exercise prescriptions would specify modality (endurance vs. resistance training), intensity zones based on VO2 max estimates and HRV, recovery windows, and even optimal time-of-day based on chronotype genetics (for example, pathways associated with PER3, CLOCK gene variants). Sleep optimization tailors wind-down protocols, light exposure schedules, and room temperature based on circadian rhythm genetics. Stress and mental health modules use genetic data of multiple pathways associated with serotonin and dopamine metabolism, and HPA axis sensitivity markers to personalize mindfulness, breathing, or cognitive behavioral approaches. Supplementation and nutraceuticals are targeted only where variants pathways generate deficiencies, insufficiencies, or elevated needs avoiding the scattershot approach of general wellness supplementation.

The system continuously learns. Interventions are treated as short experiments in real time: a change is introduced, biometric and subjective response data are tracked, and the model is updated. This generates a closed-loop system where recommendations improve with time rather than remaining static. The genetic layer is fixed, but the epigenetic and phenotypic responses it learns from are dynamic. Periodic lab panels, such as lipid panels, inflammatory markers such as CRP and IL-6, metabolic panels, hormone levels, provide ground truth checkpoints that validate or challenge the model's predictions.

Genetic data are considered highly sensitive, raising serious privacy and data security concerns even though a person's lifestyle data provides more information about disease risk or burden. The science of nutrigenomics and exercise genomics-now named precision nutrition or precision health, while promising, still has limited clinical validation for many specific gene-lifestyle interactions: the hype of the field is ahead of the evidence base in some areas. There is also the risk of genetic determinism, where people over-index on their risk scores and underestimate the power of environment and behavior. Equity is also a concern: current genomic databases are heavily skewed toward people of European ancestry, reducing accuracy for people of other backgrounds. However, statistical methods have been used to mitigate the lack of representation. For example, algorithms exist to determine local genetic ancestry around variants contributing to total- and pathway-specific polygenic risk.

Despite these limitations, the trajectory is clear: systems combining continuous biometric monitoring, whole-genome data, and adaptive AI models represent the most sophisticated version of personalized preventive medicine currently being developed.

The most rigorous framework for understanding aging biology identifies about a dozen core “hallmarks”—cellular and molecular processes that drive aging. A precision system would monitor and intervene on each one individually, based on the person's genetic predispositions.

Genomic instability is the accumulation of DNA damage over time. A genetic profile reveals how efficient the person's DNA repair machinery is-which would depend upon the collection of variants in those pathways. A subset of the contributing genes are known such as BRCA1/2, ATM, PARP1, and the various nucleotide excision repair genes involved in breast cancer. The genetically-assessed risk of these multiple pathways would dictate how aggressively to recommend antioxidant protocols, UV protection, toxin avoidance, and consumption of supplements/nutrients such as NAD+ precursors that support repair pathways. Periodic whole-genome sequencing or liquid biopsy panels can track somatic mutation burden over time as a direct aging biomarker.

Telomere attrition is considered one of the most direct clocks of cellular aging. Telomere length is partly heritable since variants in pathways that include specifically include TERT and TERC and other genes related to telomere biology may influence the person's baseline health trajectory. The system would monitor telomere length through periodic blood testing and correlate it with lifestyle factors known to slow attrition: aerobic exercise intensity and duration, chronic stress levels (telomeres are particularly sensitive to psychological stress via cortisol), sleep quality, and dietary patterns such as the Mediterranean diet. Interventions would be calibrated to the person's genetic telomere maintenance capacity.

Epigenetic drift may be the most actionable hallmark. Horvath's clock or the newer DunedinPACE epigenetic clocks measure biological age through DNA methylation patterns. They inform whether a person's biological age is ahead of or behind chronological age and estimate by how much. The system would track a person's epigenetic age through periodic blood panels and use it as the primary outcome measure for whether interventions are actually working. Methylation is highly responsive to diet (especially methyl donors such as folate, B12, choline—which are relevant to a person's MTHFR status), exercise, sleep, and stress reduction.

Loss of proteostasis refers to the decline in the cell's ability to fold, repair, and clear damaged proteins—since the accumulation of misfolded proteins is central to diseases such as Alzheimer's, Parkinson's, and many others. A person's APOE genotype is the strongest known genetic risk factor for late-onset Alzheimer's, and the system would use this to prioritize interventions that support the brain's glymphatic clearance system (deep sleep is the primary driver), upregulate autophagy through fasting protocols, and reduce neuroinflammation through omega-3 optimization and blood sugar stability.

Deregulated nutrient sensing involves pathways such as mTOR, AMPK, IGF-1, and sirtuins act as master regulators of the aging rate. Genetic variants that affect the baseline sensitivity in these pathways contribute to deregulated nutrient sensing. The system would use this information to personalize fasting protocols—time-restricted eating windows, periodic multi-day fasting mimicking protocols, or caloric restriction because these are the most potent known activators of longevity pathways that include AMPK and sirtuin activation. Individuals with naturally high IGF-1 signaling based on their genetics might receive more aggressive dietary protein modulation recommendations.

Mitochondrial dysfunction accumulates with age and is a central driver of energy decline and systemic inflammation. Variants in mitochondrial DNA itself and in nuclear gene pathways governing mitochondrial biogenesis (e.g., pathways including PGC-1α, NRF2 and connected genes) shape the person's mitochondrial aging trajectory. The system would prescribe, for example, steady-state cardio-aerobic exercise at moderate intensity (heart zone 2 out of a high of 5) which is the most evidence-backed intervention for mitochondrial biogenesis, that is calibrated specifically to heart rate variability and lactate threshold estimates.

Cellular senescence is the accumulation of zombie cells that have stopped dividing but remain metabolically active and secreting inflammatory signals (the SASP—senescence-associated secretory phenotype). This chronic low-grade inflammation, sometimes called “inflammaging,” is now recognized as a central driver of nearly every age-related disease. Genetic variants in inflammatory pathways (IL-6, TNF-α, CRP loci) determine the person's baseline inflammatory tone. The system tracks inflammatory biomarkers continuously through wearable proxies (HRV is a good real-time inflammation signal) and periodically through lab panels, using this to calibrate anti-inflammatory dietary protocols, exercise recovery windows, and potentially senolytics-which are compounds such as quercetin and dasatinib that selectively clear senescent cells, though this is still an emerging area.

In addition to tracking disease endpoints, the system reframes the goal entirely around biological age reduction or healthspan extension. It integrates multiple aging clocks into a composite score:

The epigenetic clock gives molecular age. Telomere length gives cellular replicative age. Inflammatory markers give immune age. Metabolic markers such as fasting insulin, HbA1c, and triglyceride/HDL ratio give metabolic age. Cognitive assessments give brain age. Peak VO2 max gives cardiorespiratory age. Together these form a biological age dashboard, and every lifestyle intervention is evaluated by its effect on this composite—index not just subjective wellbeing.

This is important because it transforms longevity from an abstract goal into a measurable, trackable outcome with a tight feedback loop. A person can actually see whether adopted and maintained health behaviors are effective within months, not decades.

The system would specifically identify an individual's genetic longevity profile by examining variants found enriched in centenarian populations, particularly in pathways containing or interacting with specific genes: e.g., APOE where the ε2 allele is protective, FOXO3 which is consistently associated with longevity across multiple populations, CETP which affects HDL metabolism, and SIRT1/3 in the sirtuin epigenetic pathway. Individuals with protective pathway variants may have more biological resilience where individuals with risk variant pathways may require more aggressive intervention. The system would also assess the person's cardiovascular genetic risk in detail, since heart disease and stroke remain the dominant causes of death and are among the most genetically tractable. Examples include pathways that include or interact with PCSK9 gain-of-function variants, LPA (lipoprotein a), or 9p21 risk variants (coronary artery disease) would trigger early and aggressive lipid management through diet and monitoring.

Combining these genetically-assessed, pathway-based streams, the system generates a personalized longevity protocol that might look similar to this in practice: for exercise, it prescribes a specific weekly or other periodic structure—activities such as 150-180 minutes of zone 2 cardio for mitochondrial health, two sessions of resistance training for muscle mass preservation (sarcopenia is a major driver of mortality risk), and high-intensity intervals calibrated to the person's cardiac genetics to maximize VO2 max improvement, which is one of the strongest known predictors of all-cause mortality. The exact ratios depend on the person's genetic muscle fiber composition, cardiac response genes, and inflammatory recovery capacity.

For nutrition, it moves beyond generic healthy eating to a precise protocol—specific caloric targets, protein amounts timed around resistance training (personalized by the person's IGF-1 and mTOR pathway genetics), a fasting window calibrated to the person's circadian genetics, and targeted micronutrient optimization based on the person's variants involved in absorption, utilization, and excretion. Individuals with pathways including or interacting with APOE4 gets a more aggressive saturated fat reduction protocol than individuals without it.

For sleep, it treats sleep architecture as a medical intervention rather than a lifestyle preference, given its role in glymphatic brain clearance, telomere maintenance, and epigenetic repair. It uses wearable data to identify sleep stages, cross-references with circadian genetics, and generates a precise wind-down protocol, light exposure schedule, and temperature optimization plan.

For stress physiology, given that chronic psychological stress is one of the most potent accelerators of biological aging—operating through cortisol, telomere damage, and epigenetic modification—the system monitors HRV as a real-time stress biomarker and deploys personalized recovery protocols based on HPA axis genetic sensitivity.

What makes this genuinely powerful for longevity is that these interventions are target the many interacting pathways that contribute to health of the physiological system rather than to one of the many subsystems or more reductionistic single gene variants. Reducing biological aging and expanding healthspan by one year through sleep optimization, another through exercise calibration, another through inflammatory suppression, and another through metabolic control does not add up to four years—because these pathways interact. Better mitochondrial function reduces oxidative stress which slows telomere attrition which reduces senescent cell accumulation which lowers inflammaging which improves insulin sensitivity which supports better sleep. The system models these interactions rather than treating each hallmark in isolation.

The realistic near-term goal of such a system, based on current evidence, is not radical life extension but healthspan extension, that is, compressing morbidity, maintaining cognitive and physical function into late life, and potentially shifting biological age meaningfully relative to chronological age. The science suggests that with optimal, personalized lifestyle intervention, the gap between biological and chronological age can be 10-15 years or more in either direction—which, at the population level, represents an enormous difference in quality and quantity of healthy life.

A precision disease prevention and longevity engine is the analytical and computational center of everything described above, and it is but built as a unified, continuously learning system rather than a collection of separate tools. The following is how it works at a deep level.

### Architecture Overview

The engine operates across five fundamental layers: data ingestion, biological modeling, intervention optimization, outcome prediction, and continual assessments of physiological status. These layers do not run sequentially: they operate as a continuous closed loop, updating in real time as new data arrives and outcomes are observed.

It is a living model of the body that gets more accurate over time, runs simulations of future health trajectories, and uses that to generate the most precise possible interventions for disease prevention and longevity throughout life.

### The Data Foundation

The engine requires a richer and more integrated data environment than any existing health system currently attempts.

At the genetic layer, it ideally ingests whole-genome sequencing data—but could rely on the 0.02% of the genome captured by consumer SNP arrays. It also incorporates multiomic data when available: the transcriptome (which genes are currently being expressed and at what levels), the proteome (which proteins are present and in what quantities), the metabolome (the full chemical fingerprint of a person's metabolism), and the epigenome (the methylation and histone modification patterns that control gene expression). Together these generate what systems biologists call a “deep phenotype,” a molecularly complete picture of a person's biology that goes far beyond what any single data type captures.

At the phenotypic layer, continuous wearable data streams in: HRV, sleep staging, activity, glucose, temperature, skin conductance, and increasingly, wearable biomarkers such as lactate, ketones, and inflammatory markers as sensor technology matures. Periodic clinical measurements such as comprehensive metabolic panels, specific lipid identification, hormone panels, inflammatory markers, complete blood counts, imaging that provide high-fidelity ground truth checkpoints. Biological age clocks are run regularly using epigenetic, proteomic, and composite methodologies.

At the environmental and behavioral layer, the engine ingests periodic diet and lifestyle assessments, psychological state data, social connection metrics, ancestry, culture, environmental exposures such as air quality and toxin burden, medication and supplement use data, and pathogen exposure history.

### The Biological Knowledge Graph

The engine's intelligence utilizes a continuously updated knowledge graph which is a structured representation of every known relationship between genetic variants, biological pathways, environmental exposures, interventions, and health outcomes, drawn from the entire scientific literature. Vydiant's OneHealth knowledge base models this individual level graph using multi-hop reasoning models of population level data from published scientific studies (e.g., Pubmed/PMC).

An individual's database is not static. It is a living model that incorporates new research as it is published, uses confidence-weighted relationships (so a finding replicated in ten large studies has more weight than a single small trial), and critically, understands the mechanistic links between variables rather than just statistical associations. This matters because statistical associations can mislead since the knowledge graph reasons about causal biological mechanisms, which allows it to generalize beyond what has been directly studied to predict novel interactions.

The graph encodes things such as: pathway X with variants in multiple genes reduces expression of enzyme Z by 40%, which slows pathway A, which under condition B leads to accumulation of metabolite C, which activates inflammatory signaling D, which over decades increases risk of disease E—but can be compensated by intervention F which upregulates alternative pathway G. This kind of multi-step mechanistic systems reasoning is what allows the engine to generate truly personalized recommendations rather than population-average guidelines.

### The Digital Persona Model

The computational core of the engine is a digital persona—a dynamic simulation of the person's specific physiology that runs continuously alongside an individual's real body. The digital persona is initialized from the individual's genomic and multiomic baseline and is continuously calibrated against the individual's real biometric data. It models a person's body as a system of interacting biological networks: metabolic networks governing energy and substrate utilization, signaling networks governing cellular responses to nutrients and stress, immune networks governing inflammation and pathogen defense, neuroendocrine networks governing stress hormones and circadian rhythms, and aging networks governing the hallmarks described earlier.

When the model's predictions diverge from observed outcomes such as when glucose responds differently than predicted after a dietary change causing the person's inflammation markers to spike unexpectedly, the system flags this as a signal and updates its parameters. Over months and years, it becomes increasingly accurate as a model of a specific individual rather than a generic human.

A function of the digital persona is counterfactual simulation. Before recommending an intervention, the engine runs thousands of simulations: what happens to (for example) a 10-year cardiovascular risk if there is an increase zone 2 exercise by 60 minutes per week? What is the predicted effect on epigenetic age if there is a shift to a time-restricted eating window? How does the simulated Alzheimer's risk trajectory change if deep sleep is optimized by 45 minutes per night? The engine compares intervention options not by general evidence but by their predicted effect on a person's specific biological model, then recommends the combination with the highest expected impact on the healthspan and disease prevention objectives.

### Disease Risk Modeling

Unlike standard clinical risk scores that use a handful of variables and population averages, the engine builds dynamic, individualized disease risk models for every major age-related condition.

For cardiovascular disease, it integrates total polygenic risk score (aggregating thousands of genetic variants) including specific contributing pathways, current lipid fractionation data (LDL particle size and number, not just total LDL), lipoprotein (a) levels, coronary artery calcium scoring, inflammatory markers, blood pressure patterns, metabolic status, and lifestyle data. Running these variables through a mechanistic cardiovascular model to produce a personalized risk trajectory with confidence intervals rather than a single point estimate. Crucially, it models how different interventions shift that trajectory, not just what the current risk is.

For neurodegenerative disease, it incorporates pathways that include APOE genotype, tau and amyloid biomarkers from blood (now feasible via ultrasensitive immunoassay), glymphatic efficiency estimated from sleep architecture, neuroinflammatory markers, metabolic health (insulin resistance in the brain is now recognized as a key Alzheimer's driver), and cognitive performance trajectories. It runs this through an Alzheimer's progression model to identify the preclinical curve because the pathology often begins 20 years before symptoms and what interventions at an early age have the highest evidence base for trajectory modification.

For cancer, the system integrates somatic mutation burden from liquid biopsy that assesses circulating tumor DNA in the blood as an early detection signal for clonal expansions years before clinical cancer develops. The input data includes germline cancer risk variants, immune surveillance capacity genetics, inflammatory environment, and exposure history, which generates tissue-specific cancer risk profiles and flags concerning signals for clinical follow-up.

For metabolic disease, it models insulin sensitivity trajectory, beta cell function, and fat distribution patterns through the lens of genetic metabolic architecture, predicting not just whether a person will develop type 2 diabetes but when, and what specific interventions at what intensity will most efficiently reverse the trajectory.

### The Intervention Optimization Engine

Given a biological model and disease risk landscape, the engine faces an optimization problem: given health goals, constraints, preferences, and biological response characteristics, what is the combination of adaptive lifestyle and other interventions that maximizes healthspan and minimizes disease risk most efficiently?

This is solved using reinforcement learning—an AI framework in which the system learns through trial and error which interventions produce the best outcomes for specific biology, improving its recommendations over time as it observes how the person responds. It balances multiple competing objectives simultaneously: longevity, disease prevention, cognitive performance, physical performance, metabolic health, mental health, and quality of life, weighted by personal preferences.

The engine understands that interventions interact. More aggressive time-restricted eating might amplify the benefits of resistance training for muscle preservation but could also impair sleep quality if the eating window is poorly timed relative to a person's circadian genetics. Higher intensity exercise improves cardiovascular fitness and mitochondrial health but may increase inflammatory burden beyond the person's genetic recovery capacity if volume is not carefully managed. The optimization engine models these interaction effects, and it does not recommend interventions in isolation but finds the combination that works together given a person's specific biology and constraints.

It also models biological response heterogeneity—the well-documented finding that individuals respond very differently to the same intervention. The person's genetic pathways affecting exercise response that include multiple known genes (ACTN3, ACE, PPARGC1A), dietary response (FTO, TCF7L2, APOE), and stress response (COMT, SLC6A4, NR3C1) but also unsuspected genes identified by the genetic pipeline described herein. All of these interacting pathways—that is the complete system-inform predicted response magnitudes, allowing the system to prioritize interventions where the person is a predicted high responder.

### Precision Early Detection System

One of the engine's most powerful functions is acting as a continuous, multi-modal early warning system—detecting disease signals years before clinical presentation.

It does this through anomaly detection across all data streams simultaneously. Rather than waiting for a marker to cross a clinical threshold—which in most diseases represents advanced pathology—it detects subtle directional changes across multiple markers that together signal early biological dysregulation. A slight upward drift in fasting glucose combined with a mild increase in CRP, a small decrease in HRV, and emerging changes in sleep architecture might individually be within normal ranges but collectively pattern-match to early metabolic syndrome years before any single marker would trigger clinical concern.

Liquid biopsy panels for circulating tumor DNA, cell-free DNA methylation patterns, and circulating proteins from the emerging proteomics-based early detection space (companies such as Grail, Seer, and Nautilus are building this infrastructure) would be integrated into the engine's cancer early detection module, enabling detection at stage 0 or 1 when outcomes are dramatically better.

The engine also monitors for biological age acceleration events, periods where the rate of biological aging suddenly increases, often in response to severe psychological stress, illness, or environmental exposures. These events are now detectable through epigenetic clock measurements and represent high-priority intervention windows.

### The Adaptive Intervention Protocol

The engine does not deliver a static plan. It operates on multiple timescales simultaneously:

On a real-time timescale (seconds to minutes), it monitors wearable data and provides immediate feedback—flagging when HRV indicates when a nervous system is under excessive load, when glucose response to a meal is tracking unfavorably, or when the person's sleep timing is drifting from the person's optimal circadian window.

On a weekly or other timescale, it analyzes patterns across the week's data, compares them against the previous week and against its predictions, and fine-tunes recommendations, adjusting exercise intensity, meal timing, sleep targets, and recovery protocols based on observed responses.

On a monthly or other timescale, it incorporates lab panel results, runs updated biological age assessments, evaluates whether intervention targets are being met, and makes strategic adjustments to the longer-term protocol.

On a yearly or other timescale, it re-runs comprehensive disease risk modeling with updated biomarker data, assesses trajectory changes relative to the baseline, and revises long-term intervention strategy. It also incorporates new scientific literature that may update the evidence base for specific interventions.

### The Pharmacogenomics Layer

For cases where lifestyle intervention alone is insufficient to move the needle on disease risk—particularly for individuals with high polygenic burden for specific conditions—the engine's pharmacogenomics module optimizes pharmaceutical and nutraceutical interventions.

It models drug metabolism through a person's specific CYP450 pathway profile, predicting not just whether a drug will be effective but at what dose, what side effect profile to expect, and what drug interactions to avoid based on the person's metabolic genetics. This applies both to prescription medications such as statins, antihypertensives, metformin, and to evidence-supported longevity compounds such as rapamycin, NMN/NR, berberine, and others entering clinical longevity trials.

The engine would also track the rapidly evolving space of senolytics, mTOR modulators, and epigenetic reprogramming approaches as their evidence base matures, flagging when interventions cross evidence thresholds sufficient to recommend for the person's specific risk profile.

### The Emergent Capability: Population-Level Learning

The most transformative long-term capability of such an engine, operating at scale across millions of individuals, is what it learns at the population level while preserving individual privacy.

When the engine observes that specific genetic variant combinations respond unexpectedly well to a particular intervention, better than the existing literature predicts, this signal, aggregated across thousands of individuals with that variant profile, becomes a new scientific discovery. The engine is not just applying science; it is generating it, continuously, at a scale and precision impossible with conventional clinical trials. Over time this collapses the cycle time between biological discovery and clinical application from decades to years, and eventually to months.

This is the deepest promise of a precision disease prevention and longevity engine: not just optimizing individual health trajectories but accelerating the science of human longevity itself through the continuous, real-world, high-resolution experiment of millions of people living under personalized biological optimization.

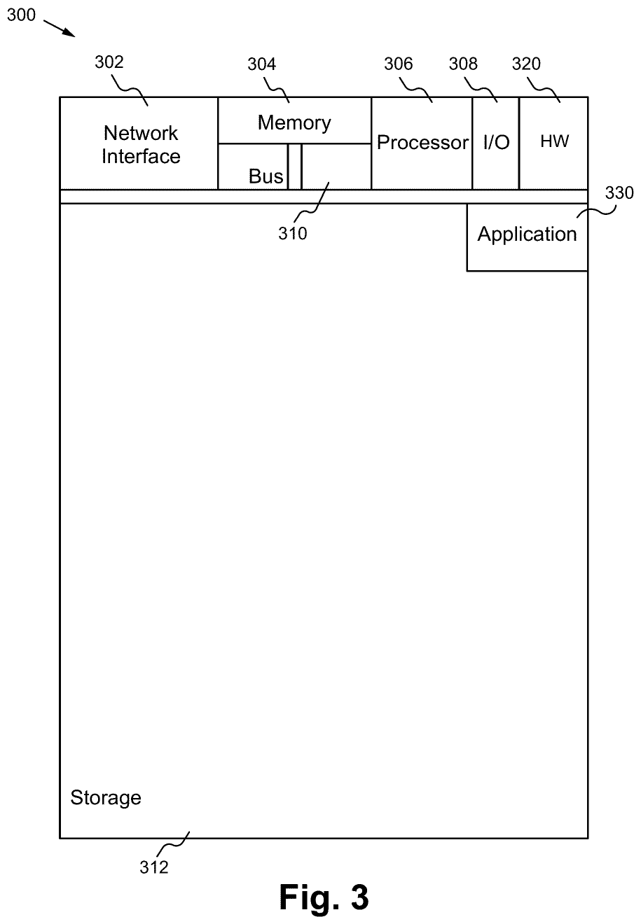

FIG. 3 illustrates a block diagram of an exemplary computing device configured to implement the precision disease prevention method according to some embodiments. The computing device 300 is able to be used to acquire, store, compute, process, communicate and/or display information. The computing device 300 is able to implement any of the precision disease prevention aspects. In general, a hardware structure suitable for implementing the computing device 300 includes a network interface 302, a memory 304, a processor 306, I/O device(s) 308, a bus 310 and a storage device 312. The choice of processor is not critical as long as a suitable processor with sufficient speed is chosen. The memory 304 is able to be any conventional computer memory known in the art. The storage device 312 is able to include a hard drive, CDROM, CDRW, DVD, DVDRW, High Definition disc/drive, ultra-HD drive, flash memory card or any other storage device. The computing device 300 is able to include one or more network interfaces 302. An example of a network interface includes a network card connected to an Ethernet or other type of LAN. The I/O device(s) 308 are able to include one or more of the following: keyboard, mouse, monitor, screen, printer, modem, touchscreen, button interface, voice, voice-enabled, and other devices. Precision disease prevention application(s) 330 used to implement the precision disease prevention method are likely to be stored in the storage device 312 and memory 304 and processed as applications are typically processed. More or fewer components shown in FIG. 3 are able to be included in the computing device 300. In some embodiments, precision disease prevention hardware 320 is included. Although the computing device 300 in FIG. 3 includes applications 330 and hardware 320 for the precision disease prevention method, the precision disease prevention method is able to be implemented on a computing device in hardware, firmware, software or any combination thereof. For example, in some embodiments, the precision disease prevention applications 330 are programmed in a memory and executed using a processor. In another example, in some embodiments, the precision disease prevention hardware 320 is programmed hardware logic including gates specifically designed to implement the precision disease prevention method.

In some embodiments, the precision disease prevention application(s) 330 include several applications and/or modules. In some embodiments, modules include one or more sub-modules as well. In some embodiments, fewer or additional modules are able to be included.

Examples of suitable computing devices include a personal computer, a laptop computer, a computer workstation, a server, a mainframe computer, a handheld computer, a quantum computer, a personal digital assistant, a cellular/mobile telephone, a smart appliance, a gaming console, a digital camera, a digital camcorder, a camera phone, a smart phone, a portable music player, a tablet computer, a mobile device, a video player, a video disc writer/player (e.g., DVD writer/player, high definition disc writer/player, ultra high definition disc writer/player), a television, a home entertainment system, an augmented reality device, a virtual reality device, smart jewelry (e.g., smart watch), a vehicle (e.g., a self-driving vehicle) or any other suitable computing device.

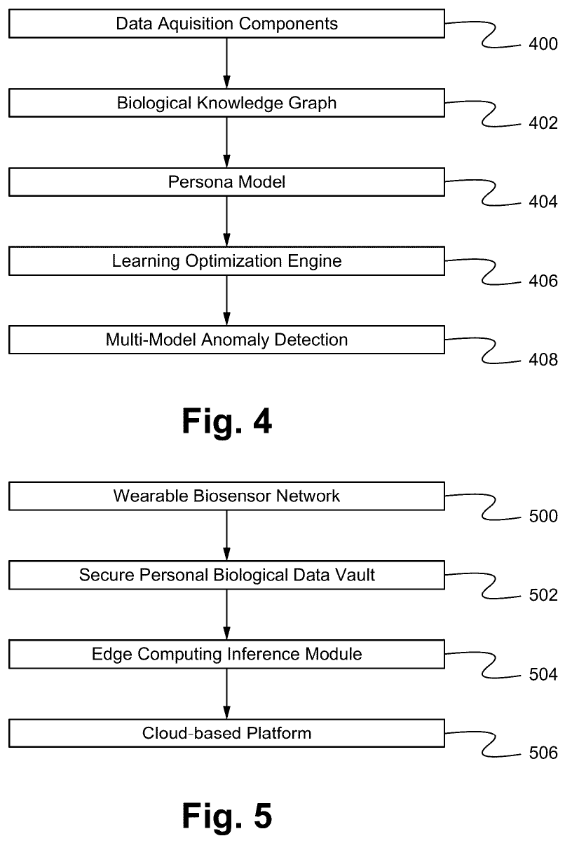

FIG. 4 illustrates a diagram of a precision disease prevention system according to some embodiments. Utilizing various data acquisition components 400, the system ingests genomic sequencing data in combination with continuous multiomic measurements and integrates these with real-time physiological monitoring via wearable biosensor arrays, periodic clinical biomarker panels, behavioral data streams, and environmental exposure measurements.

The data is processed by a causal biological knowledge graph 402 encoding mechanistic relationships between genetic variants, molecular pathways, environmental inputs, interventions, and health outcomes, derived from and continuously updated by the peer-reviewed scientific literature.

A personalized digital persona model 404 is constructed for each individual, representing a dynamic simulation of that individual's specific physiology initialized from their genomic and multiomic baseline and continuously calibrated against adaptive lifestyle, observed biometric responses, medications and health records. The digital persona model 404 executes counterfactual intervention simulations to predict individualized biological responses across a multi-dimensional intervention space encompassing nutrition, exercise, sleep, stress modulation, pharmacogenomically optimized supplementation, and emerging longevity therapeutics.

An embedded reinforcement learning optimization engine 406 identifies intervention combinations that maximally advance the individual's healthspan and disease prevention objectives, explicitly modeling biological pathway interactions and individual response heterogeneity derived from genetic variant profiles.

The system employs healthspans and biological aging—assessed through integrated epigenetic, proteomic, telomeric, and composite aging clock methodologies—as an outcome metric, replacing disease-centric diagnostic thresholds with a continuous, multi-system healthspan and aging trajectory that responds to interventions measurably prior to clinical pathology.

A multi-modal anomaly detection module 408 monitors for subclinical disease signals across all data streams simultaneously, proteomic early detection panels, and pattern deviations, given an individual's total polygenic and pathway-specific risk, enabling disease detection and intervention at preclinical stages years to decades before conventional diagnostic thresholds are crossed.

The method operates across multiple timescales simultaneously-including real-time biosensor feedback, weekly or other periodic pattern analysis, monthly or other periodic clinical integration, and annual or other periodic strategic reassessment-which generates adaptive intervention protocols that improve in precision as the individual's self-learning biological model accumulates longitudinal validation. At population scale, the system employs federated learning architecture to generate novel scientific discoveries from aggregated anonymized response data while preserving individual data sovereignty through end-to-end encryption and granular consent frameworks.

Fewer or additional components are able to be included in the system.

FIG. 5 illustrates a diagram of a precision disease prevention device architecture according to some embodiments. The device architecture encompasses a wearable biosensor network 500 for continuous physiological data acquisition, integration and analysis of clinical, family history, and electronic health records, a secure personal biological data vault 500, an edge-computing inference module 504 for real-time intervention feedback, and a cloud-based deep phenotyping, simulation and modeling platform 506. Together these constitute a closed-loop biological optimization system that converts individual genetic architecture from a static risk map into a dynamic instruction set for precision intervention, uniquely bridging the gap between molecular aging biology and individualized daily behavioral prescription in a continuously learning, causally reasoning system with no equivalent in existing medical, wellness, or research paradigms. Fewer or additional components are able to be included in the architecture.

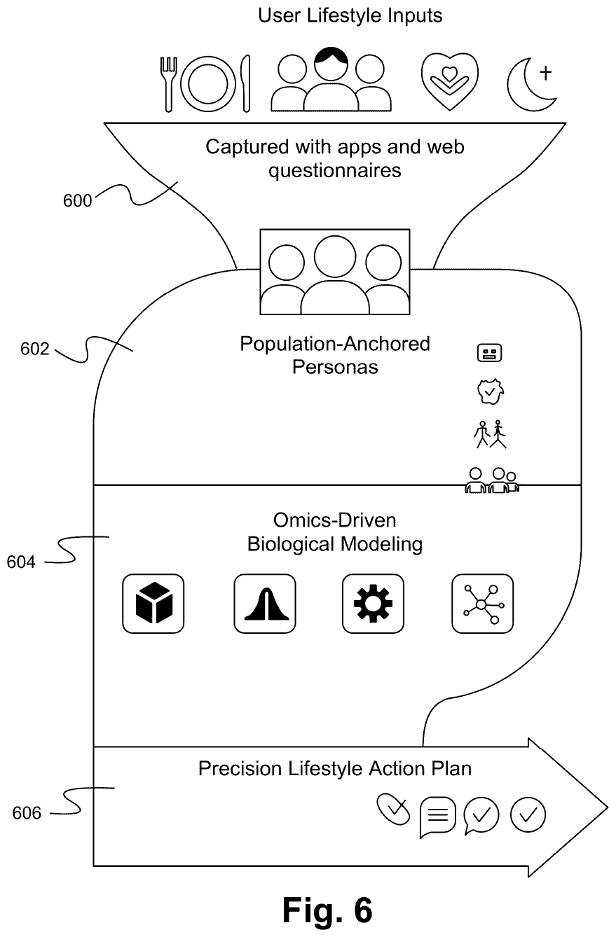

FIG. 6 illustrates a diagram of a precision disease prevention framework according to some embodiments. User lifestyle information 600 is captured with applications, web questionnaires, and other sources.

Population-anchored personas 602 are generated based on the raw user input. The input is transformed into nationally contextualized profiles. The population-anchored personas 602 are able to include nutrition personas, supplement personas, physical activity personas, social connection personas, and others. The personas 602 are able to be population-based profiles based on age, sex, ethnicity and more.

Omics-driven biological modeling 604 is performed. The omics-driven biological modeling 604 includes analytical tools. For example, the analytical tools include or generate a quality of diet score; scores for physical and social activities, stress coping, sleep; a nutrient composition engine; a vitamin sufficiency index via a vitamin sufficiency model; a biomarker normalization score; a cofactor burden score; a protein-weighted vitamin importance score via a protein-weighted vitamin impact model; pathwise genetics and a composition metabolic resilience score are able to be generated.

A precision lifestyle action plan 606 is able to be developed based on the omics-driven biological modeling 604. The precision lifestyle action plan 606 is able to provide nutrition guidance, supplement recommendations, and physical and social activity goals, as well as other information.

In operation, the system, method and device described herein make the following novel contributions to the fields of precision medicine, computational biology, and longevity science.

It is the first system to integrate whole-genome sequencing, continuous multiomic profiling, real-time physiological monitoring, lifestyle assessments, public domain environmental and social determinant of health data, and real-time adaptive intervention optimization into a unified, continuously learning closed-loop biological optimization engine. It is the first method to employ a continuously calibrated individual digital persona as the basis for personalized longevity intervention prescription. It is the first device architecture to operationalize biological age as a real-time, adaptive optimization target linked directly to intervention generation. It is the first system to bridge the gap between molecular aging biology—including the hallmarks of aging, longevity pathway modulation, and epigenetic reprogramming science—and individualized daily behavioral prescription in a causally reasoning, continuously improving system. And it is the first platform to generate novel gene-environment interaction science as a continuous, privacy-preserving byproduct of individualized biological optimization at population scale.

It is the first system to utilize AI-driven data mining to map environmental ligands/activators to transcription factors regulating genes involved in a selected disease/condition (e.g., type 2 diabetes (T2DM)) pathways, identified through published partitioned-polygenic risk analysis, identify nutrients, physical activities, and stressors that impact these molecular pathways, accounting for individual variations. Although the description uses the example of diabetes as the selected disease/condition, it is understood that other types of diseases/conditions and the related genes are able to be selected.

The system treats each person as a sample size of one. Every existing medical system, including the best personalized medicine approaches today, ultimately grounds its recommendations in population statistics. Even when a doctor says, “based on a person's risk factors,” they mean “people with a person's profile had this outcome X % of the time.” The recommendations are calibrated for the average person in a person's risk category, not for the person.

A person's genetic variants, combined with the person's continuous biometric responses to interventions, mean the system learns the person's personal dose-response curves-how the person's body specifically responds to a given sleep duration, a particular exercise intensity, a specific dietary pattern, a specific supplement, a specific drug or medication. Over time, the engine is not reasoning from population averages at all. It is reasoning from an empirical model of the person's individual biology, validated by months and years of observed responses. This is something that has never existed before: a medical system that is genuinely, deeply personalized in a biological rather than demographic sense.

It operates upstream of disease. Conventional medicine is a disease response system. Even “preventive” medicine, annual checkups, cancer screenings, cardiovascular risk scoring, is still largely designed to detect pathology early or identify high-risk individuals for monitoring. It operates at the late stages of a disease trajectory.

The system, method and device operate at an entirely different point on the timeline. It models the biological preconditions for disease: the cellular and molecular processes that, over decades, produce pathology and intervenes there. It is not asking “does this person have early-stage atherosclerosis?” but “what is this person's arterial biological aging rate, and what interventions will slow it before any lesion develops?” It is not detecting early Alzheimer's pathology; it is modeling glymphatic efficiency and neuroinflammatory tone in a 35-year-old and intervening on the trajectory before amyloid begins accumulating. This 20-to-30-year intervention window is where the real longevity gains exist, and it is territory that no current medical system is designed to operate in.

It makes biological aging an outcome metric. This is philosophically and practically transformative. Current medicine organizes itself around diseases: discrete diagnostic categories with defined treatments. A person either has hypertension or the person does not. A person either has type 2 diabetes or the person does not. This generate a system that is fundamentally reactive, because most people spend decades in subclinical states before crossing a diagnostic threshold. The system, method and device replace this with a continuous, integrated metric, e.g., biological age, that captures the overall rate and trajectory of aging across all systems simultaneously. This metric responds to interventions months or years before any disease marker would move. It integrates information from dozens of biological systems rather than evaluating them in isolation. And it generates a universal optimization target that is more meaningful than any single disease risk score, because biological age acceleration is the common upstream driver of virtually every age-related condition simultaneously. No existing medical system uses this as a primary outcome, because medicine is organized around diseases rather than the aging process that underlies them.

It learns continuously and integratively. Existing health tools such as fitness trackers, nutrition apps, and genetic testing services generate data and provide static recommendations that do not fundamentally update based on how a person's responds. Even the most sophisticated current precision medicine approaches use genetics to inform initial treatment choices but do not build a continuously improving model of the person's individual biology over time. The system, method and device are fundamentally different because they treat every intervention as an experiment, observe the outcome, and update a biological model accordingly. This means the engine gets meaningfully smarter about a person's specific biology periodically. Its intervention recommendations improve in precision over years of use in a way that has no ceiling, it never reaches a point where it has learned everything relevant about the person's individual biology, because the biology itself changes with age, environment, and experience. The compounding value of this continuous learning is unique: a system used for ten years has an extraordinarily detailed, validated model of the person's biology that simply cannot be replicated by any episodic medical interaction.

It models interactions, not just individual variables. One of the most profound limitations of current medical and wellness approaches is that they evaluate interventions in isolation. A cardiologist optimizes the person's lipids. A sleep specialist optimizes the person's sleep. A nutritionist optimizes the person's diet. An endocrinologist optimizes the person's hormones. These recommendations are generated by domain specialists operating in silos, without modeling how their recommendations interact- and biological interactions are often more important than the individual effects.

The system, method and device model a person's entire biology as an integrated system of interacting networks both internal to the body and external (e.g., environmental, lifestyle, and others). It understands that the sleep quality affects insulin sensitivity which affects inflammatory tone which affects cardiovascular risk which affects cognitive function which affects stress response which affects sleep quality. It optimizes across this entire interaction network simultaneously, finding intervention combinations that generate synergistic effects no single-domain approach could identify. The biological interactions it discovers and exploits, amplifying beneficial cascades and interrupting harmful ones, are likely where the largest longevity gains lie, and they are invisible to any system that does not model the whole simultaneously.

It converts genetic determinism into genetic agency. Consumer genetic testing, as it currently exists, is largely an exercise in genetic fatalism. A person learns about an elevated Alzheimer's risk or a high polygenic score for cardiovascular disease and the current system offers the person little beyond monitoring and anxiety. This is because existing genetic information systems cannot model the degree to which gene expression is environmentally modifiable—the critical epigenetic layer that sits between the person's DNA sequence and the actual biological outcomes.

The system, method and device fundamentally reframe this. A genome is not the person's destiny, but rather, it is a map of the person's specific biological vulnerabilities and strengths, which the engine uses to design targeted interventions that modify how those genes are expressed. Someone with APOE4 does not just receive a fatalistic Alzheimer's risk score; they receive a precision protocol specifically designed to modify every modifiable biological pathway contributing to their elevated risk such as deep sleep optimization for glymphatic clearance, specific dietary fat composition to reduce neuroinflammation, targeted aerobic exercise protocols to promote BDNF and neuroplasticity, metabolic interventions to address the insulin resistance component of Alzheimer's pathology. The genetic risk score and identification of the underlying biological pathway become an instruction set for intervention rather than a verdict. This reframing—from genetic determinism to genetic agency—is philosophically and practically unique.

It generates novel science as a byproduct. This may be the most uniquely powerful characteristic of the system operating at scale. Conventional clinical science advances through randomized controlled trials that are expensive, slow, narrow in scope, and limited in their ability to model individual variation. A PDPLE operating across millions of individuals with deep biological data and continuous outcome tracking is, in effect, running millions of simultaneous highly-instrumented natural experiments.

When the engine observes that a specific combination of genetic variants consistently shows an unexpected response to a particular intervention, that is better or worse than the existing literature predicts, this signal, validated across thousands of individuals with that profile, is a genuine scientific discovery. The engine is not just applying the existing evidence base; it is extending it continuously. It will discover gene-environment interactions, intervention synergies, and biological mechanisms that conventional research would take decades to identify. No existing medical system or research institution has ever operated with this data density, individual resolution, and continuous feedback architecture. The scientific knowledge it generates would be categorically new.

It bridges the gap between molecular biology and daily life. One of the deepest problems in translating advances in aging biology into actual human benefit is the enormous gap between what is understood at the molecular level and what reaches clinical practice. The basic science of aging—the hallmarks, the longevity pathways, the epigenetic mechanisms, is advancing rapidly. But the translation from “AMPK activation extends lifespan in model organisms” to “here is the precise, personalized exercise and fasting protocol that optimally activates AMPK in the person's specific body” does not exist in any current system. Physicians do not prescribe zone 2 cardio in precise, biologically calibrated doses. Nutritionists do not design eating protocols around the person's specific mTOR pathway genetics. Sleep specialists do not optimize deep sleep stages specifically for glymphatic amyloid clearance relative to the person's APOE genotype.

The system, method and device close this gap entirely. It takes molecular biology directly to the level of daily behavioral prescription, calibrated to the person's individual biology, updated continuously by the person's observed responses. It makes the frontier of aging science actionable in a person's daily life in a way that has never existed—not in any medical system, not in any wellness industry, not in any research setting. That translation function, operating continuously and individually, is perhaps the most uniquely valuable thing such a system could do.

What makes the Precision Disease Prevention and Longevity Engine (PDPLE) unique is not any single component but the deep integration of all of them into a unified, causally reasoning, continuously learning biological model that gets more accurate and more valuable over time.

An analogy is GPS navigation. Maps existed before GPS. Real-time traffic data existed before GPS. Route optimization algorithms existed before GPS. What GPS did was integrate them into a unified system that continuously updated, reasoned about the person's specific journey in real time, and got the person to the person's destination more efficiently than any of the components could individually. The system, method and device do the same thing for human biology—and the destination it navigates toward is a longer, healthier life with fundamentally less disease.

That synthesis—deep, real-time, causal, individualized, continuously improving—is what makes it unique. Nothing like it currently exists in medicine, wellness, or research. It represents a genuinely new category.

As described herein, the system, method and device utilize genome, lifestyle and wearables information, a causal knowledge graph, a digital persona model, a reinforcement learning intervention engine, and generating precision lifestyle protocols.

Important aspects of the system, method and device include: genetic pathway-specific intervention modeling; transcription factor→ligand→lifestyle mapping; closed-loop biomarker feedback; reinforcement-learning intervention optimization; and healthspan trajectory modeling.

The system, method and device implement features for longevity and healthspan modeling such as PRS, lifestyle knowledge graphs, real-time biometrics, reinforcement learning, and intervention optimization.

The present invention has been described in terms of specific embodiments incorporating details to facilitate the understanding of principles of construction and operation of the invention. Such reference herein to specific embodiments and details thereof is not intended to limit the scope of the claims appended hereto. It will be readily apparent to one skilled in the art that other various modifications may be made in the embodiment chosen for illustration without departing from the spirit and scope of the invention as defined by the claims.

What is claimed is:

1. A system comprising:
a plurality of data acquisition components configured to acquire genomic sequencing or assays of selected variants data, continuous multiomic measurements, and real-time physiological measurements;
a causal biological knowledge graph configured for encoding mechanistic relationships between genetic variants, molecular pathways, environmental inputs, interventions, and health outcomes;
a personalized digital persona model configured to represent a dynamic simulation of a person's specific physiology initialized from a genomic and multiomic baseline and continuously calibrated against adaptive lifestyle, observed biometric responses, medications and health records; and
an embedded reinforcement learning optimization engine configured to identify intervention combinations that maximally advance the individual's healthspan and disease prevention objectives, explicitly modeling biological pathway interactions and individual response heterogeneity derived from genetic variant profiles.

2. The system of claim 1 wherein the real-time physiological measurements are acquired via wearable biosensor arrays, periodic clinical biomarker panels, behavioral data streams and environmental exposure measurements.

3. The system of claim 1 wherein the causal biological knowledge graph is derived from and continuously updated by scientific literature.

4. The system of claim 1 wherein the digital persona model is further configured to execute counterfactual intervention simulations to predict individualized biological responses across a multi-dimensional intervention space encompassing nutrition, exercise, sleep, stress modulation, pharmacogenomically optimized supplementation, drugs (prescription and non-prescription) and emerging longevity therapeutics.

5. The system of claim 1 further comprising a multi-modal anomaly detection module configured to monitor for subclinical disease signals across all data streams simultaneously, proteomic detection panels, and pattern deviations, given an individual's total polygenic and pathway-specific risk, enabling disease detection and intervention at preclinical stages.

6. The system of claim 1 wherein the system operates across multiple timescales simultaneously with real-time biosensor feedback, weekly or other periodic pattern analysis, monthly or other periodic clinical integration, and annual or other periodic strategic reassessment, generating adaptive intervention protocols that improve in precision as an individual biological model accumulates longitudinal validation.

7. A method programmed in a non-transitory memory of a device, the method comprising:
acquiring genomic sequencing or assays of selected variants data, continuous multiomic measurements, and real-time physiological measurements with a plurality of data acquisition components;
encoding mechanistic relationships between genetic variants, molecular pathways, environmental inputs, interventions, and health outcomes with a causal biological knowledge graph;
representing a dynamic simulation of a person's specific physiology initialized from a genomic and multiomic baseline and continuously calibrated against adaptive lifestyle, observed biometric responses, medications and health records with a personalized digital persona model; and
identifying intervention combinations that maximally advance the individual's healthspan and disease prevention objectives, explicitly modeling biological pathway interactions and individual response heterogeneity derived from genetic variant profiles with an embedded reinforcement learning optimization engine.

8. The method of claim 7 wherein the real-time physiological measurements are acquired via wearable biosensor arrays, periodic clinical biomarker panels, behavioral data streams and environmental exposure measurements.

9. The method of claim 7 wherein the causal biological knowledge graph is derived from and continuously updated by scientific literature.

10. The method of claim 7 wherein the digital persona model is further configured to execute counterfactual intervention simulations to predict individualized biological responses across a multi-dimensional intervention space encompassing nutrition, exercise, sleep, stress modulation, pharmacogenomically optimized supplementation, drugs (prescription and non-prescription), and emerging longevity therapeutics.

11. The method of claim 7 further comprising monitoring for subclinical disease signals across all data streams simultaneously, proteomic detection panels, and pattern deviations, given an individual's total polygenic and pathway-specific risk, enabling disease detection and intervention at preclinical stages with a multi-modal anomaly detection module.

12. The method of claim 7 wherein the method operates across multiple timescales simultaneously with real-time biosensor feedback, weekly or other periodic pattern analysis, monthly or other periodic clinical integration, and annual or other periodic strategic reassessment, generating adaptive intervention protocols that improve in precision as an individual biological model accumulates longitudinal validation.

13. A device comprising:
a non-transitory memory configured for storing an application, the application configured for:
receiving genomic sequencing or assays of selected variants data, continuous multiomic measurements, and real-time physiological measurements from a plurality of data acquisition components;
encoding mechanistic relationships between genetic variants, molecular pathways, environmental inputs, interventions, and health outcomes with a causal biological knowledge graph;
representing a dynamic simulation of a person's specific physiology initialized from a genomic and multiomic baseline and continuously calibrated against adaptive lifestyle, observed biometric responses, medications and health records with a personalized digital persona model; and
identifying intervention combinations that maximally advance the individual's healthspan and disease prevention objectives, explicitly modeling biological pathway interactions and individual response heterogeneity derived from genetic variant profiles with an embedded reinforcement learning optimization engine; and
a processor configured for processing the application.

14. The device of claim 13 wherein the real-time physiological measurements are acquired via wearable biosensor arrays, periodic clinical biomarker panels, behavioral data streams and environmental exposure measurements.

15. The device of claim 13 wherein the causal biological knowledge graph is derived from and continuously updated by scientific literature.

16. The device of claim 13 wherein the digital persona model is further configured to execute counterfactual intervention simulations to predict individualized biological responses across a multi-dimensional intervention space encompassing nutrition, exercise, sleep, stress modulation, pharmacogenomically optimized supplementation, drugs (prescription and non-prescription), and emerging longevity therapeutics.

17. The device of claim 13 wherein the application is configured for monitoring for subclinical disease signals across all data streams simultaneously, proteomic detection panels, and pattern deviations, given an individual's total polygenic and pathway-specific risk, enabling disease detection and intervention at preclinical stages with a multi-modal anomaly detection module.

18. The device of claim 13 wherein the method operates across multiple timescales simultaneously with real-time biosensor feedback, weekly or other periodic pattern analysis, monthly or other periodic clinical integration, and annual or other periodic strategic reassessment, generating adaptive intervention protocols that improve in precision as an individual biological model accumulates longitudinal validation.

19. A system comprising:
a wearable biosensor network configured for continuous physiological data acquisition, integration and analysis of medical and family history and electronic health records;
a secure personal biological data vault;
an edge-computing inference module configured for real-time intervention feedback; and
a cloud-based deep phenotyping, simulation and modeling platform.

20. A method programmed in a non-transitory memory of a device, the method comprising:
selecting condition-associated genes;
retrieving promoter regions;
identifying Transcription Factor (TF) binding sites in the promotor regions;
identifying ligands that bind to TFs;
linking TF ligands to nutritional components;
linking the TF ligands to physical activity;
linking the TF ligands to emotional stress;
building a TF-ligand-lifestyle database;
developing an AI Model for novel TF-lifestyle predictions;
performing clinical validation using biomarkers and wearables; and
implementing AI explainability for TF-lifestyle predictions.

21. The method of claim 20 wherein studies and literature are mined to identify relevant genes.

22. The method of claim 20 wherein the promotor regions are extracted using genomic databases.

23. The method of claim 20 wherein Chromatin ImmunoPrecipitation sequencing (ChIP-seq) and motif-based analysis are used to detect TF binding in promoter regions.

24. The method of claim 20 wherein databases are queried for experimentally validated TF-ligand interactions.

25. The method of claim 20 wherein identifying food sources containing TF ligands using food compound databases.

26. The method of claim 20 wherein exercise-induced molecular changes that regulate TF activity are identified.

27. The method of claim 20 wherein stress-related hormones and neurotransmitters affecting TF activity are identified.

28. The method of claim 20 wherein a machine learning model integrating gene expression, ligand structure, and lifestyle impact is trained.

29. The method of claim 20 wherein AI predictions are validated using real-world biomarker data such as Continuous Glucose Monitor (CGM), Heart Rate Variability (HRV), and blood biomarkers.
